(19) United States
(12) Patent Application Publication
(10) Pub. No.: US 2021/0325367 A1
(43) Pub. Date: Oct. 21, 2021

(54) Controlled Exposure to Pathogens for Generating Immunity

(71) Applicant: Satish Mahna, Beachwood, OH (US)

(72) Inventor: Satish Mahna, Beachwood, OH (US)

(21) Appl. No.: 16/896,969

(22) Filed: Jun. 9, 2020

Related U.S. Application Data
(60) Provisional application No. 63/012,790, filed on Apr. 20, 2020.

(51) Int. Cl. G01N 33/49; G01N 33/537; A61K 35/16

(52) U.S. Cl. CPC G01N 33/49; A61K 2035/124; A61K 35/16; G01N 33/537

(57) ABSTRACT

A method generates a natural immunity to a pathogen in the absence of a vaccine. The process draws a blood sample, exposes the blood sample to a pathogen outside of a living organism, and measures the antibody type, level, and a pathogen level in the exposed blood sample. The method injects the blood sample exposed to the pathogen into the source of the blood sample when one or more antibody types are detected at a predetermined level and the pathogen level is below a predetermined level.

### PRIORITY CLAIM

The present application claims the benefit of priority under 35 U.S. § 119 from U.S. Provisional Patent Application No. 63/012,790, entitled “Controlled Exposure to Pathogens for Generating Immunity,” filed on Apr. 20, 2020, the disclosure of which is hereby incorporated by reference in its entirety for all purposes.

### BACKGROUND OF THE DISCLOSURE

### Technical Field

This patent application relates to mediating epidemics and pandemics, and specifically to generating natural treatments for rapidly developing and extensive infections.

### Related Art

Vaccines are effective in preventing infectious diseases. The injections of live, attenuated or inactivated pathogens induce a protective immunity that defends the body against invaders that cause infectious diseases. Typically, the protection lasts for years or a lifetime. For passive immunity, plasma of a convalescent patient may be injected into a recipient.

While vaccines are highly effective, their development also takes years leaving most individuals vulnerable to life-threatening diseases. Generally, vaccines are generated in three phases. The first phase determines the genetic sequence of the disease. The second phase processes active and/or passive parts of the disease to produce vaccination candidates. The third phase implements clinical trials that measure vaccine candidates' safety and effectiveness in creating immune responses and preventing infection. Once successful, vaccines are mass produced. Some vaccines are effective at first, but do not provide lasting immunity because of mutations or because of the waning of persons' immune response.

While the process can be sped up in response to pandemics, it is usually controlled to prevent adverse reactions. Vaccines that are rushed can cause significant side effects later. Poorly developed vaccines can cause infections and also cause symptoms that are worse than when individuals are not inoculated. When designed for widespread use, even a fast track development process is relatively slow, deliberate, peer-reviewed, and evidence-based to minimize errors. This is even more true in societies that are skeptical of vaccines.

### SUMMARY

According to certain aspects of the present disclosure, a method of generating a natural immunity to an infectious disease in the absence of a vaccine is provided. The method includes drawing a blood sample from a source. The method also includes separating the blood sample into white blood cells and plasma. The method also includes exposing the white blood cells and the plasma that are separated from the blood sample to a pathogen in vitro. The method also includes measuring an antibody type, an antibody level, and a pathogen level in the plasma exposed to the pathogen. The method also includes injecting a portion of both the white blood cells and the plasma exposed to the pathogen into the source from whom the blood sample was drawn when a predetermined antibody type is detected at a first predetermined threshold and the pathogen level is below a second predetermined level.

According to certain aspects of the present disclosure, a method of generating a natural immunity to an infectious disease in the absence of a vaccine is provided. The method includes drawing a blood sample from a source. The method also includes separating the blood sample into white blood cells and plasma. The method also includes exposing, in vitro, the white blood cells and the plasma that are separated from the blood sample to a pathogen, wherein the pathogen is inactivated. The method also includes measuring an antibody type and an antibody level in the plasma exposed to the pathogen. The method also includes injecting a portion of both the white blood cells and the plasma exposed to the inactivated pathogen into the source from whom the blood sample was drawn when the antibody level is measured at a first predetermined antibody level threshold.

According to certain aspects of the present disclosure, a method of generating a natural immunity to an infectious disease in the absence of a vaccine is provided. The method includes drawing a blood sample from a source. The method also includes separating the blood sample into white blood cells and plasma. The method also includes exposing, in vitro, the white blood cells and the plasma that are separated from the blood sample to a pathogen. The method also includes measuring an antibody type, an antibody level, and a pathogen level in the plasma exposed to the pathogen. The method also includes injecting a portion of both the white blood cells and the plasma exposed to the pathogen into the source from whom the blood sample was drawn when the antibody level is measured at a predetermined antibody level threshold and the pathogen level is below a predetermined pathogen level threshold. The method also includes monitoring, after injecting the portion of both the white blood cells and the plasma exposed to the pathogen into the source, reactions of the source.

### BRIEF DESCRIPTION OF THE DRAWINGS

The accompanying drawings are included to provide further understanding and are incorporated in, and constitute, a part of this specification. The accompanying drawings illustrate disclosed embodiments and together with the description serve to explain the principles of the disclosed embodiments. In the drawings:

FIG. 1 is a process for developing a natural immunity against infectious diseases.

FIG. 2 is a second process for developing a natural immunity against infectious diseases.

FIG. 3 is a third process for developing a natural immunity against infectious diseases.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

Natural immunity makes it hard for diseases to spread. Individuals are protected from infection because they are surrounded by others who are immune. When effective widespread vaccinations are unavailable, individuals who fall ill and recover from the disease can provide individual and widespread protection. To be effective, this means that many individuals must be infected and recover to reach a widespread immunity (e.g., Herd Immunity). The disclosed processes minimize the human cost of infecting large communities by providing a resistance to an infection associated with one or more pathogens without directly exposing individuals to the pathogens. The processes minimize the risk of severe illness and even death. The process exposes an individual's plasma and white blood cells separated from the blood sample drawn to the live, attenuated or inactivated (e.g., inactivated by heat or ultraviolet radiation) pathogen in vitro. For example, plasma and white blood cells separated from the blood sample drawn are exposed to the pathogen in vitro in labware such as, but not limited to, test tubes, flasks, Petri dishes, microtiter plates, and other labware well-known in the industry. The plasma and white blood cells separated from the blood sample drawn and that are exposed to the pathogen is processed in vitro and then injected into the individual from whom the blood was drawn. Some systems may measure the level and the type of antibodies in the blood, after exposure to the pathogen in vitro and, before it is injected into the individual. To optimize the process, the concentration/amount of the pathogen (virus or other pathogens) introduced into the medium (such as plasm containing white blood cells), in vitro, can be titrated to generate maximum antibody production (e.g., Dose Response Curve). Similarly, duration of the in vitro incubation period (e.g. 24 hours or 48 hours or 72 hours and so on) can also be optimized to generate adequate levels of antibody production before injecting the medium (e.g. Plasma now containing white blood cells, neutralized pathogen and antibodies produced) back into the person from whom blood was drawn.

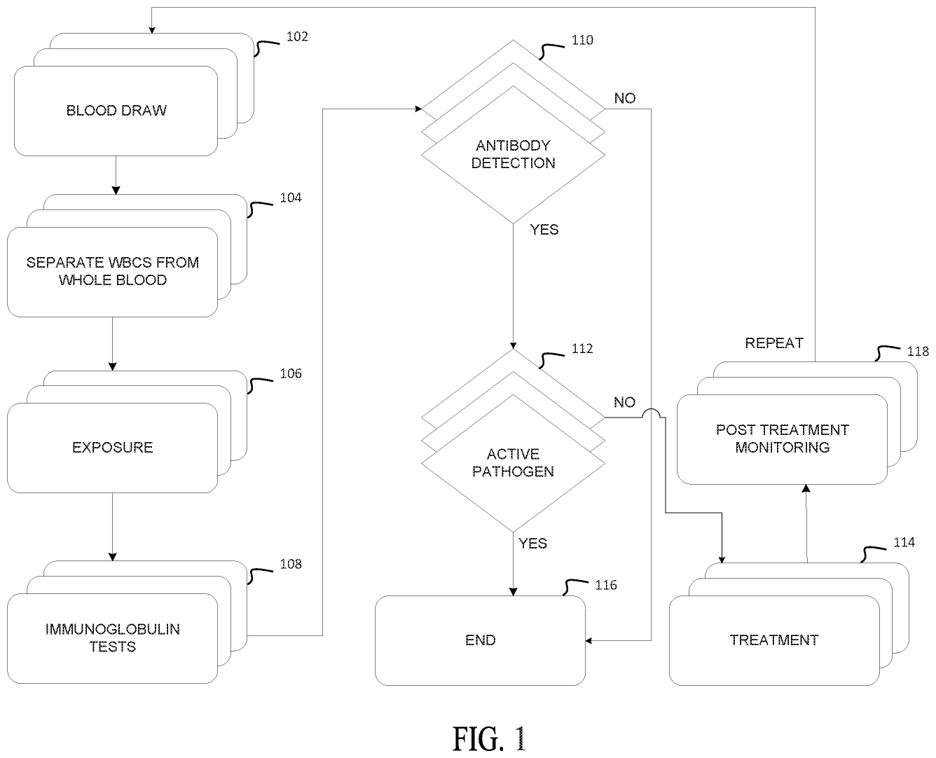

The process begins in FIG. 1 by drawing blood from an individual (e.g., a source) at 102. From that sample, white blood cells and plasma are separated at 104 and kept in a medium such as plasma, for example. The white blood cells and plasma that are separated (and which are harvested outside of the living organism and placed in vitro) are exposed to a pathogen (which may be live, attenuated or inactivated by heat or ultraviolet radiation) causing a disease at 106. When exposed, the white blood cells process the antigen (Pathogen, such as virus or bacteria) and initiate immune responses. Should the pathogen be in the process of mutation (such as a mutating virus), the immune response of the white blood cells also alters and changes in response to the changes in the disease. In some cases, even when the mutations occur after the controlled exposure to the pathogen (such as virus) was completed, the person so immunized may remain protected from the mutated pathogen (virus) since there may be patches of the original pathogen (virus) that remain unchanged. In some processes, the cells processing the antigen (pathogen, such as virus) and generating immune responses include neutrophils, monocytes, macrophages and/or lymphocytes, for example.

In FIG. 1, the white blood cells exposed to the antigen (pathogen, such as virus), and the medium in which they are kept, are tested for any active pathogen and antibody level at 108 and 110. In certain aspects, testing for any active pathogen activity may not be necessary if the pathogen was attenuated or inactivated with heat or ultraviolet radiation. If no active pathogen is detected in the medium at 112 or if it is below a predetermined level/threshold, and there is confirmation of sufficient level of one or more desired antibodies present in the medium, the activated white blood cells and the medium (e.g., plasma containing antibodies) that were exposed to the pathogen are injected into the person from whom the blood was drawn as treatment, as depicted at 114, and the individual (e.g., the source) is thereafter monitored, at 118, for reactions and/or immune response as well as to any potential adverse effects. After receiving the white blood cells and plasma that was exposed to the pathogen, the individual, from whom the blood sample was drawn, is deemed protected when adequate levels of antibodies against the pathogen are detected in the individual's blood (e.g., determining that a second antibody level of a second blood sample of the source is at a second predetermined antibody level threshold). Such an individual can donate plasma, after receiving (e.g., injected with) a booster dose of the active or attenuated pathogen to enhance the antibody level, to another individual who has developed an active disease from that pathogen. If a substantial level of active pathogens is detected at 112, the process terminates at 116. To confirm its effectiveness, the process (e.g., injections of the activated white blood cells and the medium) can be repeated in other (limited number of) people and/or other clinical trials, and if found safe and effective, is repeated on a larger scale population.

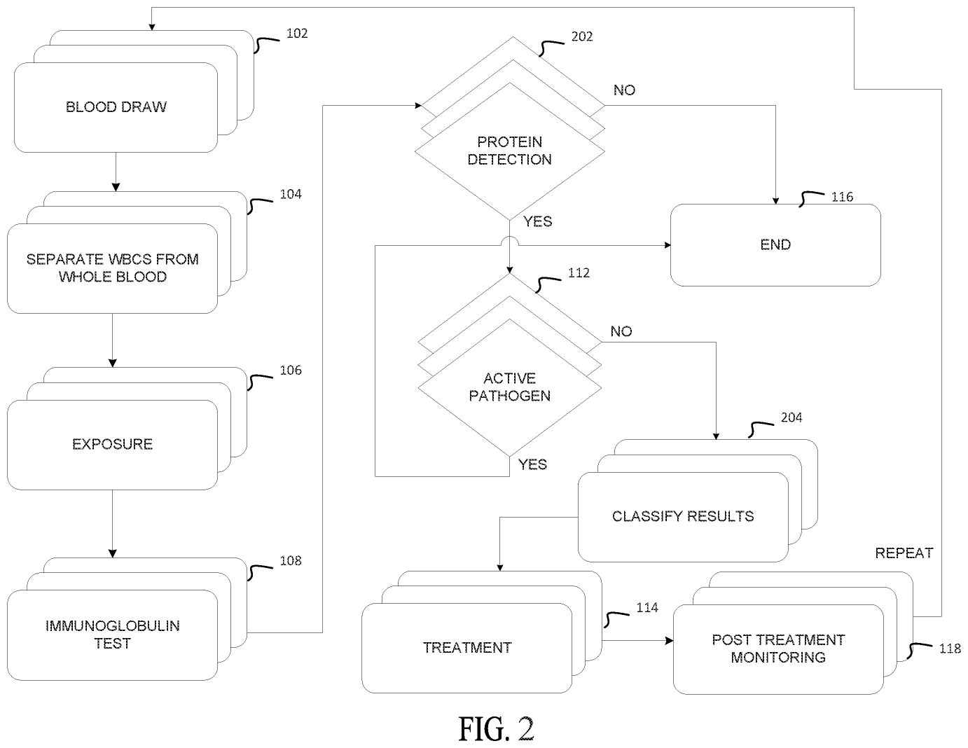

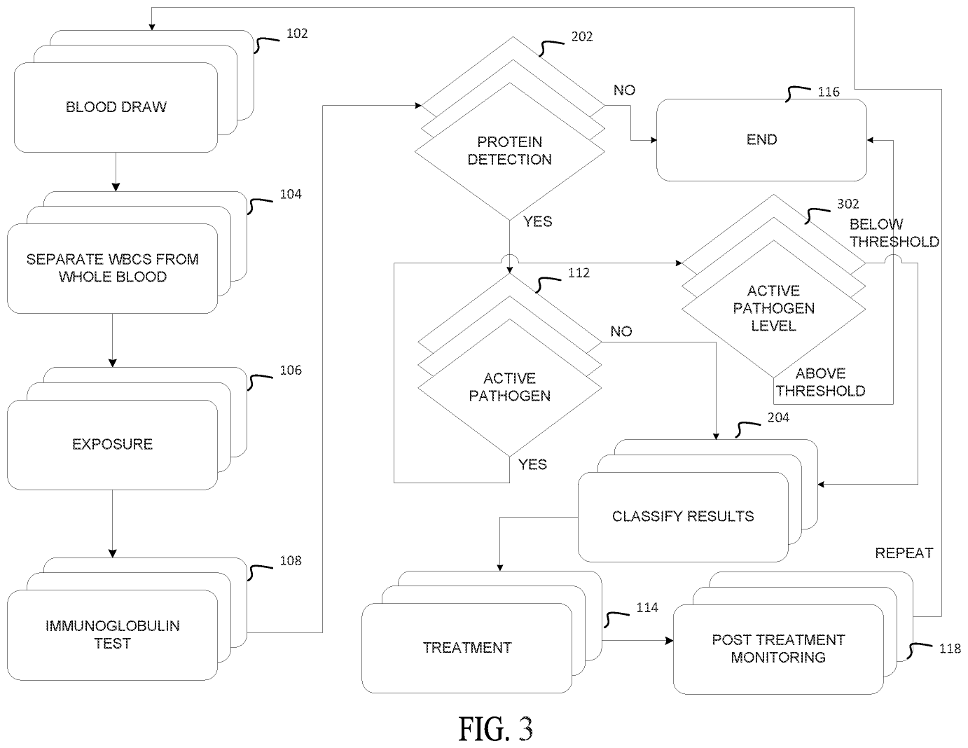

In FIGS. 2 and 3, the antibodies are detected through a sensitive antibody assay at 202 and treatment at 114 is based on the type and level of antibody isotypes detected (e.g., exceeding a predetermined level/threshold) at 204. In FIG. 3, the treatment 114 may include a heavily weakened/attenuated or inactivated strain of the pathogen that will not cause harm. The treatment 114 may include an attenuated version of the pathogen that is determined at 302.

Other systems, methods, features and advantages will be, or will become, apparent to one with skill in the art upon examination of the figures and detailed description. It is intended that all such additional systems, methods, features and advantages be included within this description, be within the scope of the disclosure, and be protected by the following claims.

1. A method of stimulating immune cells ex vivo to generate a autologous passive immune response in a human patient, to a predetermined infectious disease in the absence of a vaccine, in a human patient who has not been previously exposed to the predetermined infectious disease, the method comprising the steps of:
drawing a predetermined amount of blood comprising white blood cells and plasma from the human patient to obtain a blood sample;
separating the white blood cells and the plasma from the blood sample to obtain a treatment sample that comprises both the white blood cells and the plasma;
determining if the treatment sample was previously exposed to a certain pathogen known to cause the predetermined infectious disease by measuring for a preexisting pathogen level for that certain pathogen, a preexisting antibody level for that certain pathogen, or a combination of the two, in the treatment sample;
if the treatment sample has not previously been exposed to the certain pathogen, exposing the treatment sample to an amount of the certain pathogen in vitro sufficient to produce in the treatment sample an amount of antibodies capable of generating an immune response to the predetermined infectious disease;
measuring the antibody type and the antibody level in the exposed treatment sample;
determining if the antibody type and the antibody level are present in an amount sufficient to meet a first predetermined threshold;
if the antibody type and the antibody level measured meets the first predetermined threshold, injecting a portion of the exposed treatment sample into the human patient from whom the blood sample was drawn in an amount sufficient to provide the human patient with immunity to the predetermined infectious disease.

2. The method of claim 1, further comprising the step of: titrating a predetermined concentration of the certain pathogen, in vitro, before the treatment sample is exposed to the certain pathogen.

3. The method of claim 1, further comprising the step of: incubating the exposed treatment sample for a predetermined incubation period before the antibody type and level are measured.

4. The method of claim 1, wherein the step of exposing the treatment sample to the amount of certain pathogen in vitro is performed in a labware.

5. The method of claim 4, wherein the labware is selected from the group consisting of a test tube, a flask, a Petri dish, and a microtiter plate.

6. The method of claim 1, wherein the amount of the certain pathogen comprises a live pathogen.

7. The method of claim 1, further comprising the step of: after injecting the portion of the exposed treatment sample into the human patient, monitoring reactions of the human patient to the exposed treatment sample.

8. The method of claim 1, wherein the step of measuring the antibody level comprises measuring antibody levels of a plurality of antibody isotype types.

9. The method of claim 1, wherein the amount of the certain pathogen comprises an inactivated pathogen.

10. The method of claim 9, wherein the amount of the certain pathogen is inactivated with heat.

11. The method of claim 9, wherein the amount of the certain pathogen is inactivated with ultraviolet radiation.

12. The method of claim 9, further comprising the step of: after injecting the portion of the exposed treatment sample into the human patient, monitoring reactions of the human patient to the exposed treatment sample.

13. The method of claim 9, further comprising the step of: titrating a predetermined concentration of the amount of the certain inactivated pathogen, in vitro, before the treatment sample is exposed to the certain inactivated sample.

14. The method of claim 9, further comprising: incubating the exposed treatment sample for a predetermined incubation period before the antibody type and antibody level are measured.

15. The method of claim 9, wherein the step of exposing the treatment sample to the inactivated pathogen in vitro is performed in a labware.

16. The method of claim 15, wherein the labware is selected from the group consisting of a test tube, a flask, a Petri dish, and a microtiter plate.

17. (canceled)

18. The method of claim 21, further comprising the step of:
after injecting the portion of the exposed treatment sample into the human patient and monitoring the reactions to the exposed treatment sample, taking a second blood sample from the human patient and measuring a second antibody level of the second blood sample; and
if the second antibody level of the second blood sample meets a second predetermined threshold, injecting the human patient with a booster dose of the certain pathogen.

19. The method of claim 21, further comprising the step of:
titrating a predetermined concentration of the amount of the certain pathogen, in vitro, before the amount of the certain pathogen is exposed to the treatment sample.

20. The method of claim 21, further comprising the step of:
incubating the exposed treatment sample for a predetermined incubation period before the antibody type and antibody level are measured.

21. A method of generating a stimulating immune cells ex vivo to generate a autologous passive immune response in a human patient to a predetermined infectious disease in the absence of a vaccine, in a human patient who has not been previously exposed to the predetermined infectious disease, the method comprising the steps of:
drawing a predetermined amount of blood comprising white blood cells and plasma from the human patient to obtain a blood sample;
separating the white blood cells and the plasma from the blood sample to obtain a treatment sample comprising both the white blood cells and the plasma;
determining if the treatment sample was previously exposed to a certain pathogen known to cause the predetermined infectious disease by measuring a pathogen level for that certain pathogen;
if the treatment sample was not previously exposed to the certain pathogen, exposing the treatment sample to an amount of the certain pathogen in vitro sufficient to produce an amount of antibodies capable of generating an immune response to the predetermined infectious disease; wherein the amount of antibodies produces an antibody type and an antibody level in the exposed treatment sample;
measuring the antibody type and the antibody level in the exposed treatment sample;
determining if the antibody type and the antibody level are present in an amount sufficient to meet a first predetermined threshold;
if the first predetermined threshold is met, injecting a portion of the exposed treatment sample into the human patient from whom the blood sample was drawn in an amount sufficient to provide the human patient with immunity to the predetermined infectious; and
after injecting the portion of the exposed treatment sample into the human patient, monitoring reactions of the human patient to the exposed treatment sample.
